(19) United States
(12) Patent Application Publication
(10) Pub. No.: US 2020/0214650 A1
(43) Pub. Date: Jul. 9, 2020

(54) ELECTRONIC DEVICE FOR MEASURING BIOMETRIC INFORMATION AND OPERATION METHOD THEREOF

(71) Applicant: Samsung Electronics Co., Ltd., Gyeonggi-do (KR)

(72) Inventors: Jae-Myeon LEE, Gyeonggi-do (KR); Jae-Suk CHOI, Gyeonggi-do (KR); Jae-Il KIM, Gyeonggi-do (KR); Nam-Hoon KIM, Gyeonggi-do (KR); Tae-Ho KIM, Chungcheongbuk-do (KR); Jeong-Min PARK, Gyeonggi-do (KR); Seung-Eun LEE, Seoul (KR)

(21) Appl. No.: 16/614,945

(22) PCT Filed: May 25, 2018
(86) PCT No.: PCT/KR2018/005985

§ 371 Date: Nov. 19, 2019

(30) Foreign Application Priority Data
May 25, 2017 (KR) 10-2017-0064837

(51) Int. Cl. A61B 5/00; A61B 5/1455; A61B 5/021; A61B 5/024

(52) U.S. Cl. CPC A61B 5/7475; A61B 5/1455; A61B 2562/0238; A61B 5/02433; A61B 5/7435; A61B 5/021

(57) ABSTRACT

An electronic device according to various embodiments of the present invention may comprise a sensor for acquiring a biometric signal, and a processor, wherein the processor is configured to: acquire a biometric signal by using the sensor; measure first biometric information at least on the basis of a first portion of the biometric signal; measure second biometric information at least on the basis of a second portion that is at least a part of the first portion; and display at least one of the first biometric information and the second biometric information using a display functionally connected to the electronic device.

### TECHNICAL FIELD

Various embodiments of the disclosure relate to an electronic device for measuring biometric information and an operating method thereof.

### BACKGROUND ART

Recently, electronic devices including sensors capable of measuring biometric information of a user have been developed. A user may recognize his or her body state by measuring information related to his or her body by using an electronic device.

An electronic device may measure various pieces of biometric information, including a heart rate, an oxygen saturation, stress, blood pressure, and the like of a user, by using a sensor. For example, an electronic device may sense a user's body part by using a sensor. The electronic device may measure various pieces of biometric information of the user by using sensing information acquired through the sensor.

### DETAILED DESCRIPTION OF THE INVENTION

### Technical Problem

A user needs separate measurement processes according to various pieces of biometric information in order to measure various pieces of biometric information, including a heart rate, an oxygen saturation, stress, blood pressure, and the like. That is, an electronic device cannot simultaneously measure multiple pieces of biometric information through one measurement process. Further, in order to measure biometric information of the user, the user's body part needs to come in contact with a sensor or maintain a state, in which the biometric information can be acquired, during a predetermined period of time. Therefore, inconvenience is caused to the user while the user measures various pieces of biometric information by using the electronic device.

Various embodiments of the disclosure may provide an electronic device capable of simultaneously measuring multiple pieces of biometric information on the basis of a biometric signal acquired using a sensor and an operating method thereof.

### Technical Solution

In accordance with an aspect of the disclosure, an electronic device may include: a sensor configured to acquire a biometric signal; and a processor, wherein the processor is configured to: acquire a biometric signal by using the sensor; measure first biometric information at least on the basis of a first portion of the biometric signal; measure second biometric information at least on the basis of a second portion which is at least a part of the first portion; and display at least one piece of information among the first biometric information and the second biometric information, on a display functionally connected to the electronic device.

In accordance with another aspect of the disclosure, an operating method of an electronic device may include: acquiring a biometric signal by using a sensor of the electronic device; measuring first biometric information at least on the basis of a first portion of the biometric signal, and measuring second biometric information at least on the basis of a second portion which is at least a part of the first portion; and displaying at least one piece of information among the first biometric information and the second biometric information, on a display functionally connected to the electronic device.

### Advantageous Effects

An electronic device according to various embodiments of the disclosure can simultaneously measure multiple pieces of biometric information on the basis of one biometric signal acquired using one sensor.

### BRIEF DESCRIPTION OF DRAWINGS

A detailed description of each of the drawings will be provided to more fully understand the drawings to which the detailed description of the disclosure refers.

FIG. 1 illustrates a block diagram of an electronic device and a network according to various embodiments of the disclosure.

FIG. 2 is a block diagram of an electronic device according to various embodiments.

FIG. 3 is a block diagram of a program module according to various embodiments.

FIG. 4A is a rough block diagram of an electronic device according to various embodiments of the disclosure.

FIG. 4B is a block diagram for explaining an operation of the processor illustrated in FIG. 4A.

FIG. 5A is a flowchart for explaining an operation of an electronic device according to various embodiments of the disclosure.

FIG. 5B is a graph for explaining the operation of the electronic device of FIG. 5A.

FIG. 6 is a flowchart for explaining an operation of an electronic device according to various embodiments of the disclosure.

FIG. 7 is a flowchart for explaining an operation of an electronic device according to various embodiments of the disclosure.

FIG. 8 is a flowchart for explaining an operation of an electronic device according to various embodiments of the disclosure.

FIG. 9 is a flowchart for explaining an operation of an electronic device according to various embodiments of the disclosure.

FIG. 10A is a flowchart for explaining an operation of an electronic device according to various embodiments of the disclosure.

FIG. 10B is a flowchart for explaining an operation of an electronic device according to various embodiments of the disclosure.

FIG. 11 is a flowchart for explaining an operation of an electronic device according to various embodiments of the disclosure.

FIG. 12A is a flowchart for explaining an operation of an electronic device according to various embodiments of the disclosure.

FIG. 12B is a graph for explaining the operation of the electronic device of FIG. 12A.

FIG. 13 is a flowchart for explaining an operation of an electronic device according to various embodiments of the disclosure.

FIG. 14A is a flowchart for explaining an operation of an electronic device according to various embodiments of the disclosure.

FIG. 14B is a graph for explaining the operation of the electronic device of FIG. 14A.

FIGS. 15A, 15B, 15C, 15D, and 15E each illustrate a user interface provided by an electronic device according to various embodiments of the disclosure.

### MODE FOR CARRYING OUT THE INVENTION

Hereinafter, various embodiments of the disclosure will be described with reference to the accompanying drawings. The embodiments and the terms used therein are not intended to limit the technology disclosed herein to specific forms, and should be understood to include various modifications, equivalents, and/or alternatives to the corresponding embodiments. In describing the drawings, similar reference numerals may be used to designate similar elements. A singular expression may include a plural expression unless the context clearly indicates otherwise. In the disclosure, the expression “A or B” or “at least one of A and/or B” may include all possible combinations of items enumerated together. The expression “a first”, “a second”, “the first”, or “the second” may modify corresponding elements regardless of the order or the importance thereof, and are used merely to distinguish each element from the others without unduly limiting the elements. When an element (e.g., first element) is referred to as being “(functionally or communicatively) connected”, or “coupled” to another element (second element), the element may be connected directly to said another element or connected to the another element through yet another element (e.g., third element).

In the disclosure, the expression “configured to” may be used interchangeably with, for example, “suitable for”, “having the capacity to”, “adapted to”, “made to”, “capable of”, or “designed to” in terms of hardware or software, according to circumstances. In some situations, the expression “device configured to” may mean that the device, together with other devices or components, “is able to”. For example, the phrase “processor adapted (or configured) to perform A, B, and C” may mean a dedicated processor (e.g., embedded processor) only for performing the corresponding operations or a general-purpose processor (e.g., Central Processing Unit (CPU) or Application Processor (AP)) that can perform the corresponding operations by executing one or more software programs stored in a memory device.

An electronic device according to various embodiments of the disclosure may include at least one of, for example, a smart phone, a tablet Personal Computer (PC), a mobile phone, a video phone, an electronic book reader (e-book reader), a desktop PC, a laptop PC, a netbook computer, a workstation, a server, a Personal Digital Assistant (PDA), a Portable Multimedia Player (PMP), an MPEG-1 audio layer-3 (MP3) player, a medical device, a camera, and a wearable device. The wearable device may include at least one of an accessory type (e.g., a watch, a ring, a bracelet, an anklet, a necklace, a pair of glasses, a contact lens, or a Head-Mounted Device (HMD)), a fabric or clothing integrated type (e.g., an electronic clothing), a body-mounted type (e.g., a skin pad, or tattoo), and a bio-implantable type (e.g., an implantable circuit). In some embodiments, the electronic device may include at least one of, for example, a television, a Digital Video Disk (DVD) player, an audio player, a refrigerator, an air conditioner, a vacuum cleaner, an oven, a microwave oven, a washing machine, an air cleaner, a set-top box, a home automation control panel, a security control panel, a media box (e.g., Samsung HomeSync™, Apple TV™, or Google TV™), a game console (e.g., Xbox™ and PlayStation™), an electronic dictionary, an electronic key, a camcorder, and an electronic photo frame.

In other embodiments, the electronic device may include at least one of various medical devices (e.g., various portable medical measuring devices (a blood glucose monitoring device, a heart rate monitoring device, a blood pressure measuring device, a body temperature measuring device, etc.), a Magnetic Resonance Angiography (MRA), a Magnetic Resonance Imaging (MRI), a Computed Tomography (CT) machine, a scanning machine, and an ultrasonic machine), a navigation device, a Global Navigation Satellite System (GNSS), an Event Data Recorder (EDR), a Flight Data Recorder (FDR), a Vehicle Infotainment Device, electronic devices for a ship (e.g., a navigation device for a ship, a gyro-compass, etc.), avionics, a security device, an automotive head unit, a robot for home or industry, a drone, an Automatic Teller's Machine (ATM) in banks, a Point Of Sale (POS) device in a shop, and Internet-of-things devices (e.g., a light bulb, various sensors, a sprinkler device, a fire alarm, a thermostat, a streetlamp, a toaster, athletic equipment, a hot water tank, a heater, a boiler, etc.). According to some embodiments, an electronic device may include at least one of furniture, a part of a building/structure or car, an electronic board, an electronic signature receiving device, a projector, and various types of measuring instruments (e.g., a water meter, an electric meter, a gas meter, a radio wave meter, etc.). In various embodiments, the electronic device may be flexible, or may be a combination of two or more of the above-described various devices. The electronic device according to embodiments of the disclosure is not limited to the above-described devices. In the disclosure, the term “user” may indicate a person who uses an electronic device or a device (e.g., an artificial intelligence electronic device) using an electronic device.

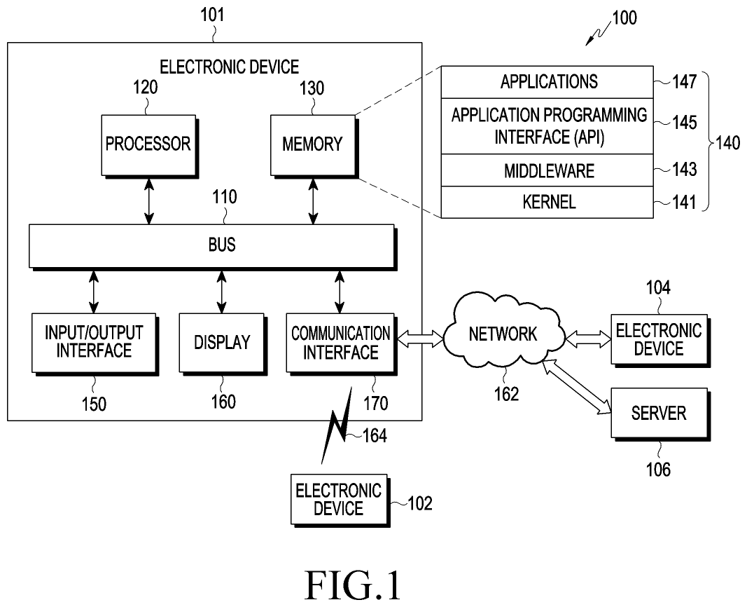

An electronic device 101 within a network environment 100 according to various embodiments will be described with reference to FIG. 1. The electronic device 101 may include a bus 110, a processor 120, a memory 130, an input/output interface 150, a display 160, and a communication interface 170. In some embodiments, at least one of the elements of the electronic device 100 may be omitted therefrom, or the electronic device 100 may further include other elements. The bus 110 may include a circuit configured to interconnect the elements 110 to 170 and deliver communication (e.g., a control message or data) between the elements. The processor 120 may include one or more of a Central Processing Unit (CPU), an Application Processor (AP), and a Communication Processor (CP). The processor 120, for example, may be configured to execute operations or data processing related to the control and/or communication of at least one other element of the electronic device 101.

The memory 130 may include a volatile and/or non-volatile memory. The memory 130 may be configured to store, for example, instructions or data related to at least one other element of the electronic device 101. According to an embodiment, the memory 130 may store software and/or a program 140. The program 140 may include, for example, a kernel 141, middleware 143, an Application Programming Interface (API) 145, and/or application programs (or “applications”) 147. At least some of the kernel 141, the middleware 143, and the API 145 may be referred to as an “Operating System (OS)”. The kernel 141 may control or manage, for example, system resources (e.g., the bus 110, the processor 120, and the memory 130) used to execute operations or functions implemented by other programs (e.g., the middleware 143, the API 145, and the application programs 147). Also, the kernel 141 may provide an interface through which the middleware 143, the API 145, or the application programs 147 may access the individual elements of the electronic device 101 so as to control or manage the system resources.

The middleware 143 may serve as, for example, an intermediary that enables the API 145 or the application programs 147 to communicate with the kernel 141 to exchange data. Also, the middleware 143 may process one or more task requests received from the application programs 147 according to the priorities of the task requests. For example, the middleware 143 may assign priorities which allow use of the system resources (e.g., the bus 110, the processor 120, the memory 130, etc.) of the electronic device 101 to one or more of the application programs 147, and may process the one or more task requests. The API 145 is an interface through which the applications 147 control functions provided by the kernel 141 or the middleware 143, and may include, for example, at least one interface or function (e.g., instruction) for file control, window control, image processing, text control, and the like. The input/output interface 150, for example, may be configured to deliver, to the other element(s) of the electronic device 101, commands or data input from a user or a different external device. Alternatively, the input/output interface 150 may be configured to output, to the user or the different external device, commands or data received from the other element(s) of the electronic device 101.

Examples of the display 160 may include a Liquid Crystal Display (LCD), a Light-Emitting Diode (LED) display, an Organic Light-Emitting Diode (OLED) display, a MicroElectroMechanical Systems (MEMS) display, and an electronic paper display. The display 160 may display, for example, various types of content (e.g., text, images, videos, icons, symbols, etc.) to a user. The display 160 may include a touch screen, and may receive, for example, a touch, gesture, proximity, or hovering input using an electronic pen or a part of a user's body. The communication interface 170 may be configured to establish, for example, communication between the electronic device 101 and an external device (e.g., a first external electronic device 102, a second external electronic device 104, or a server 106). For example, the communication interface 170 may be configured to be connected to a network 162 through wireless communication or wired communication so as to communicate with the external device (e.g., the second external electronic device 104 or the server 106).

The wireless communication may use, for example, at least one of Long-Term Evolution (LTE), LTE-Advance (LTE-A), Code Division Multiple Access (CDMA), Wideband CDMA (WCDMA), Universal Mobile Telecommunications System (UMTS), Wireless Broadband (WiBro), Global System for Mobile communications (GSM), or the like, as a cellular communication protocol. According to an embodiment, the wireless communication may include, for example, at least one of Wi-Fi, Light Fidelity (LF), Bluetooth, Bluetooth Low Energy (BLE), Zigbee, Near Field Communication (NFC), magnetic secure transmission, Radio Frequency (RF), or Body Area Network (BAN), as illustrated as an example of short-range communication 164 in FIG. 1. According to an embodiment, the wireless communication may include Global Navigation Satellite System (GNSS). The GNSS may include, for example, a Global Positioning System (GPS), a Global Navigation Satellite System (Glonass), a Beidou Navigation Satellite System (hereinafter, “Beidou”), or a European Global Satellite-based Navigation System (Galileo). Hereinafter, the “GPS” may be interchangeably used herein with the “GNSS”. The wired communication may include, for example, at least one of a Universal Serial Bus (USB), a High Definition Multimedia Interface (HDMI), Recommended Standard 232 (RS-232), power line communication, a Plain Old Telephone Service (POTS), or the like. The network 162 may include at least one of a telecommunication network such as a computer network (e.g., a LAN or a WAN), the Internet, or a telephone network.

Each of the first and second external electronic devices 102 and 104 may be of a type identical to, or different from, that of the electronic device 101. According to various embodiments, all or some of the operations executed in the electronic device 101 may be executed in another electronic device or multiple electronic devices (e.g., the electronic devices 102 and 104 or the server 106). According to an embodiment, when the electronic device 101 has to perform some functions or services automatically or in response to a request, the electronic device 101 may request another device (e.g., the electronic device 102 or 104 or the server 106) to execute at least some functions relating thereto, instead of, or in addition to, executing the functions or services by itself. Said other electronic device (e.g., the electronic device 102 or 104 or the server 106) may execute the requested functions or the additional functions and may deliver an execution result to the electronic device 101. The electronic device 101 may process the received result as it is or additionally so as to provide the requested functions or services. To this end, cloud computing, distributed computing, or client-server computing technology may be used.

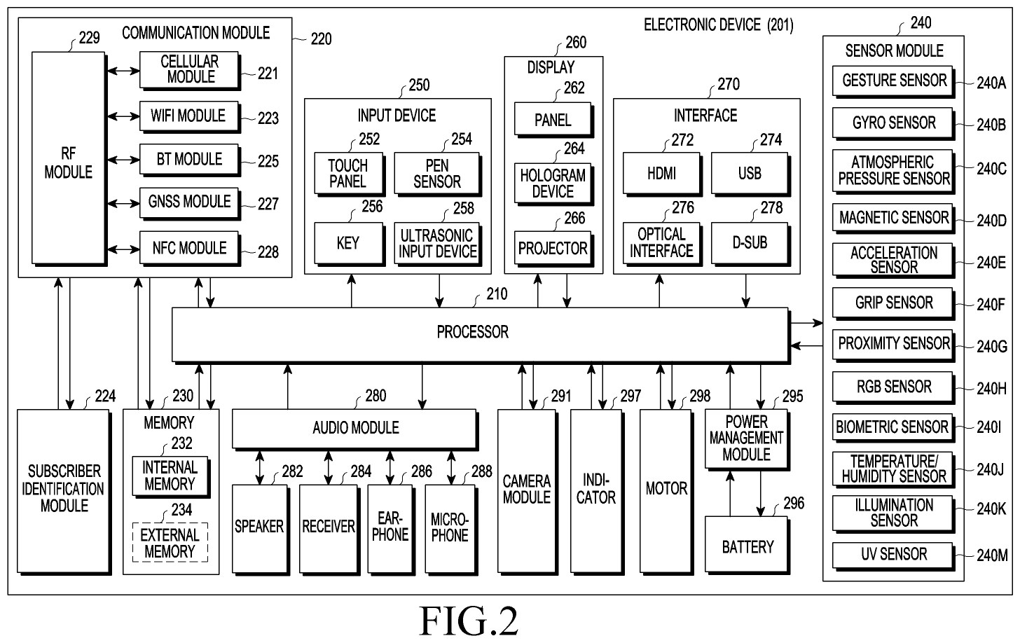

FIG. 2 is a block diagram illustrating an electronic device 201 according to various embodiments. The electronic device 201 may include, for example, the entirety, or a part, of the electronic device 101 illustrated in FIG. 1. The electronic device 201 may include at least one processor (e.g., an AP) 210, a communication module 220, a subscriber identification module 224, a memory 230, a sensor module 240, an input apparatus 250, a display 260, an interface 270, an audio module 280, a camera module 291, a power management module 295, a battery 296, an indicator 297, and a motor 298. The processor 210, for example, may be configured to drive an operating system or application programs to control multiple hardware or software elements connected thereto, and perform various types of data processing and operations. The processor 210 may be implemented by, for example, a System on Chip (SoC). According to an embodiment, the processor 210 may further include a Graphic Processing Unit (GPU) and/or an image signal processor. The processor 210 may include at least some (e.g., a cellular module 221) of the elements illustrated in FIG. 2. The processor 210 may load, into a volatile memory, commands or data received from at least one of the other elements (e.g., a non-volatile memory) to process the same, and may store resulting data in the non-volatile memory.

The communication module 220 may have a configuration identical or similar to that of the communication interface 170 illustrated in FIG. 1. The communication module 220 may include, for example, the cellular module 221, a Wi-Fi module 223, a Bluetooth module 225, a GNSS module 227, an NFC module 228, and an RF module 229. The cellular module 221 may provide, for example, a voice call, a video call, a text message service, an Internet service, and the like through a communication network. According to an embodiment, the cellular module 221 may identify and authenticate the electronic device 201 within a communication network by using the subscriber identification module (e.g., a SIM card) 224. According to an embodiment, the cellular module 221 may perform at least some of the functions that the processor 210 may provide. According to an embodiment, the cellular module 221 may include a Communication Processor (CP). According to some embodiments, at least some (e.g., two or more) of the cellular module 221, the Wi-Fi module 223, the Bluetooth module 225, the GNSS module 227, or the NFC module 228 may be included in one Integrated Chip (IC) or IC package. The RF module 229 may transmit or receive, for example, a communication signal (e.g., an RF signal). The RF module 229 may include, for example, a transceiver, a Power Amplifier Module (PAM), a frequency filter, a Low Noise Amplifier (LNA), an antenna, or the like. According to another embodiment, at least one of the cellular module 221, the Wi-Fi module 223, the Bluetooth module 225, the GNSS module 227, or the NFC module 228 may transmit or receive an RF signal through a separate RF module. The subscriber identification module 224 may include, for example, a card or an embedded SIM including a subscriber identification module, and may include unique identify information (e.g., an Integrated Circuit Card Identifier (ICCID)) or subscriber information (e.g., an International Mobile Subscriber Identity (IMSI)).

The memory 230 (e.g., the memory 130) may include, for example, an internal memory 232 or an external memory 234. The internal memory 232 may include, for example, at least one of: a volatile memory (e.g., a Dynamic Random Access Memory (DRAM), a Static RAM (SRAM), or a Synchronous DRAM (SDRAM)); and a nonvolatile memory (e.g., a One-Time Programmable Read Only Memory (OTPROM), a Programmable ROM (PROM), an Erasable and Programmable ROM (EPROM), an Electrically Erasable and Programmable ROM (EEPROM), a mask ROM, a flash ROM, a flash memory, a hard drive, or a Solid-State Drive (SSD)). The external memory 234 may include a flash drive, for example, Compact Flash (CF), Secure Digital (SD), Micro Secure Digital (Micro-SD), Mini Secure Digital (Mini-SD), extreme Digital (xD), a Multi-Media Card (MMC), or a memory stick. The external memory 234 may be functionally or physically connected to the electronic device 201 through various interfaces.

The sensor module 240 may, for example, measure a physical quantity or detect the operating state of the electronic device 201 and may convert the measured or detected information into an electrical signal. The sensor module 240 may include, for example, at least one of a gesture sensor 240A, a gyro sensor 240B, an atmospheric pressure sensor 240C, a magnetic sensor 240D, an acceleration sensor 240E, a grip sensor 240F, a proximity sensor 240G a color sensor 240H (e.g., a Red, Green, and Blue (RGB) sensor), a biometric sensor 2401, a temperature/humidity sensor 240J, an illuminance sensor 240K, or an Ultraviolet (UV) sensor 240M. Additionally or alternatively, the sensor module 240 may include, for example, an E-nose sensor, an electromyography (EMG) sensor, an electroencephalogram (EEG) sensor, an electrocardiogram (ECG) sensor, an infrared (IR) sensor, an iris sensor, and/or a fingerprint sensor. The sensor module 240 may further include a control circuit configured to control at least one sensor included therein. In some embodiments, the electronic device 201 may further include a processor configured to control the sensor module 240 as a part of the processor 210 or separately from the processor 210, so as to control the sensor module 240 while the processor 210 is in a sleep state.

The input apparatus 250 may include, for example, a touch panel 252, a (digital) pen sensor 254, a key 256, or an ultrasonic input unit 258. The touch panel 252 may use, for example, at least one of a capacitive method, a resistive method, an infrared method, or an ultrasonic method. Also, the touch panel 252 may further include a control circuit. The touch panel 252 may further include a tactile layer to provide, to a user, a tactile reaction. The (digital) pen sensor 254 may include, for example, a recognition sheet that is a part of the touch panel or is separate from the touch panel. The key 256 may include, for example, a physical button, an optical key, or a keypad. The ultrasonic input unit 258 may detect an ultrasonic wave generated by an input tool through a microphone (e.g., a microphone 288), and may check data corresponding to the detected ultrasonic wave.

The display 260 (e.g., the display 160) may include a panel 262, a hologram apparatus 264, a projector 266, and/or a control circuit configured to control them. The panel 262 may be implemented to be, for example, flexible, transparent, or wearable. The panel 262, together with the touch panel 252, may be implemented as at least one module. According to an embodiment, the panel 262 may include a pressure sensor (or force sensor) capable of measuring the strength of a pressure by the user's touch. The pressure sensor may be implemented in a single body with the touch panel 252, or may be implemented by one or more sensors separate from the touch panel 252. The hologram apparatus 264 may show a three-dimensional image in the air by using an interference of light. The projector 266 may display an image by projecting light onto a screen. The screen may be, for example, located inside or outside of the electronic device 201. The interface 270 may include, for example, a High-Definition Multimedia Interface (HDMI) 272, a Universal Serial Bus (USB) 274, an optical interface 276, or a D-subminiature (D-sub) 278. The interface 270 may be included, for example, in the communication interface 170 illustrated in FIG. 1. Additionally or alternatively, the interface 270 may include, for example, a Mobile High-definition Link (MHL) interface, a Secure Digital (SD) card/Multi-Media Card (MMC) interface, or an Infrared Data association (IrDA) standard interface.

The audio module 280 may convert, for example, a sound signal into an electrical signal, and vice versa. At least some elements of the audio module 280 may be included, for example, in the input/output interface 150 illustrated in FIG. 1. The audio module 280 may process sound information that is input or output through, for example, a speaker 282, a receiver 284, an earphone 286, the microphone 288, or the like. The camera module 291 is, for example, a device capable of capturing a still image and a moving image. According to an embodiment, the camera module 291 may include one or more image sensors (e.g., a front sensor or a rear sensor), a lens, an Image Signal Processor (ISP), or a flash (e.g., an LED or a xenon lamp). The power management module 295 may manage, for example, power of the electronic device 201. According to an embodiment, the power management module 295 may include a Power Management Integrated Circuit (PMIC), a charger Integrated Circuit (IC), or a battery or fuel gauge. The PMIC may have a wired and/or wireless charging method. Examples of the wireless charging method may include a magnetic resonance method, a magnetic induction method, an electromagnetic wave method, and the like, and an additional circuit, such as a coil loop, a resonance circuit, or a rectifier, may be further included for wireless charging. The battery gauge may measure, for example, a residual quantity of the battery 296, and a voltage, current, or temperature thereof while the battery is charged. The battery 296 may include, for example, a rechargeable battery and/or a solar battery.

The indicator 297 may indicate a particular state (e.g., a booting state, a message state, or a charging state) of the electronic device 201 or a part (e.g., the processor 210) thereof. The motor 298 may convert an electrical signal into a mechanical vibration and may generate a vibration, a haptic effect, and the like. The electronic device 201 may include a mobile TV supporting device (e.g., a GPU) capable of processing media data according to, for example, Digital Multimedia Broadcasting (DMB), Digital Video Broadcasting (DVB), or mediaFlo™ standards. Each of the above-described elements according to the disclosure may include one or more components, and the names of the corresponding elements may vary with the type of electronic device. In various embodiments, some elements may be omitted from the electronic device (e.g., the electronic device 201) or additional elements may be further included therein, or some of the elements may be combined into a single entity that may perform functions identical to those of the relevant elements before being combined.

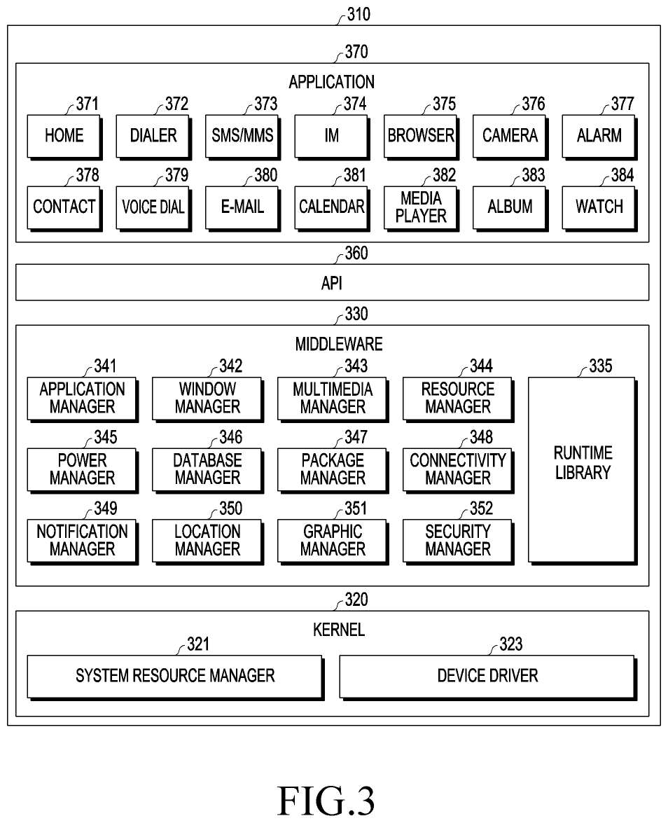

FIG. 3 is a block diagram illustrating a program module according to various embodiments. According to an embodiment, the program module 310 (e.g., the program 140) may include an operating system that controls resources related to an electronic device (e.g., the electronic device 101) and/or various applications (e.g., the application programs 147) executed on the operating system. The operating system may be, for example, Android™, iOS™, Windows™, Symbian™, Tizen™, or Bada™. Referring to FIG. 3, the program module 310 may include a kernel 320 (e.g., the kernel 141), middleware 330 (e.g., the middleware 143), an API 360 (e.g., the API 145), and/or an application 370 (e.g., the application program 147). At least a part of the program module 310 may be preloaded on the electronic device, or may be downloaded from an external electronic device (e.g., the electronic device 102 or 104 or the server 106).

The kernel 320 may include, for example, a system resource manager 321 and/or a device driver 323. The system resource manager 321 may control, allocate, or retrieve system resources. According to an embodiment, the system resource manager 321 may include a process manager, a memory manager, or a file system manager. The device driver 323 may include, for example, a display driver, a camera driver, a Bluetooth driver, a shared-memory driver, a USB driver, a keypad driver, a Wi-Fi driver, an audio driver, or an Inter-Process Communication (IPCm) driver. The middleware 330 may provide, for example, a function which the application 370 needs in common, or may provide various functions to the application 370 through the API 360 so that the application 370 can use limited system resources in the electronic device. According to an embodiment, the middleware 330 may include, for example, at least one of a runtime library 335, an application manager 341, a window manager 342, a multimedia manager 343, a resource manager 344, a power manager 345, a database manager 346, a package manager 347, a connectivity manager 348, a notification manager 349, a location manager 350, a graphic manager 351, or a security manager 352.

The runtime library 335 may include, for example, a library module used by a compiler to add a new function through a programming language while the applications 370 are executed. The runtime library 335 may perform input/output management, memory management, or arithmetic function processing. The application manager 341 may manage, for example, the life cycle of the application 370. The window manager 342 may manage GUI resources used on a screen. The multimedia manager 343 may detect a format necessary to reproduce media files, and may encode or decode media files by using a coder/decoder (codec) appropriate for the relevant format. The resource manager 344 may manage a source code or memory space of the application 370. The power manager 345 may manage, for example, the capacity of a battery or power, and may provide power information necessary for an operation of an electronic device. According to an embodiment, the power manager 345 may interwork with a Basic Input/Output System (BIOS). The database manager 346 may, for example, generate, search, or change a database to be used in the applications 370. The package manager 347 may manage installation or update of an application distributed in the form of a package file.

The connectivity manager 348 may manage, for example, wireless connectivity. The notification manager 349 may provide a user with an event, for example, arrival message, promise, or proximity notification. The location manager 350 may manage, for example, location information of the electronic device. The graphic manager 351 may manage, for example, a graphic effect to be provided to a user and a user interface related thereto. The security manager 352 may provide, for example, system security or user authentication. According to an embodiment, the middleware 330 may include a telephony manager for managing a voice or video call function of the electronic device or a middleware module capable of forming a combination of the functions of the above-described elements. According to an embodiment, the middleware 330 may provide a module specialized according to the type of operating system. The middleware 330 may dynamically remove some of the existing elements, or may add new elements thereto. The API 360 may be a set of, for example, API programming functions and may have different configurations depending on operating systems. For example, in the case of Android or iOS, one API set may be provided for each platform, and in the case of Tizen, two or more API sets may be provided for each platform.

The application 370 may include an application that provides, for example, a home 371, a dialer 372, an SMS/MMS 373, an Instant Message (IM) 374, a browser 375, a camera 376, an alarm 377, a contact 378, a voice dial 379, an e-mail 380, a calendar 381, a media player 382, an album 383, and a watch 384, health care (e.g., measuring an exercise quantity or blood sugar), or environmental information (e.g., atmospheric pressure, humidity, or temperature information). According to an embodiment, the application 370 may include an information exchange application capable of supporting information exchange between the electronic device and an external electronic device. Examples of the information exchange application may include a notification relay application for delivering particular information to the external electronic device, and a device management application for managing the external electronic device. For example, the notification relay application may deliver notification information generated by another application of the electronic device to the external electronic device, or may receive notification information from the external electronic device and provide the received notification information to the user. For example, the device management application may install, delete, or update a function (e.g., turning-on/turning-off the external electronic device itself (or some elements) or adjusting the brightness (or resolution) of the display) of the external electronic device communicating with the electronic device or an application operating on the external electronic device. According to an embodiment, the application 370 may include an application (e.g., a health-care application of a mobile medical device) designated according to an attribute of the external electronic device. According to an embodiment, the application 370 may include an application received from the external electronic device. At least a part of the program module 310 may be implemented (e.g., executed) in software, firmware, hardware (e.g., the processor 210), or as a combination of at least two or more thereof, and may include a module, program, routine, instruction set, or process for performing one or more functions.

The term “module” as used herein may include a unit consisting of hardware, software, or firmware, and may, for example, be used interchangeably with the term “logic”, “logical block”, “component”, “circuit”, or the like. The “module” may be an integrated component, or a minimum unit for performing one or more functions or a part thereof. The “module” may be mechanically or electronically implemented and may include, for example, an Application-Specific Integrated Circuit (ASIC) chip, a Field-Programmable Gate Array (FPGA), or a programmable-logic device, which performs certain operations and has been known or is to be developed in the future.

At least some of devices (e.g., modules or functions thereof) or methods (e.g., operations) according to various embodiments may be implemented by an instruction which is stored a computer-readable storage medium (e.g., the memory 130) in the form of a program module. When the instruction is executed by a processor (e.g., the processor 120), the processor may perform a function corresponding to the instruction. Examples of the computer-readable storage medium may include a hard disk, a floppy disk, a magnetic medium (e.g., a magnetic tape), an optical media (e.g., CD-ROM, DVD), a magneto-optical media (e.g., a floptical disk), an inner memory, and the like. The instruction may include a code made by a compiler or a code executable by an interpreter. A module or a program module according to various embodiments may include one or more of the above-described components or may further include other additional components, or some of the above-described components may be omitted therefrom. The operations performed by modules, program modules, or other elements according to various embodiments may be performed in a sequential, parallel, repetitive, or heuristic manner, and at least some of the operations may be performed in different orders or omitted, or other operations may be added thereto.

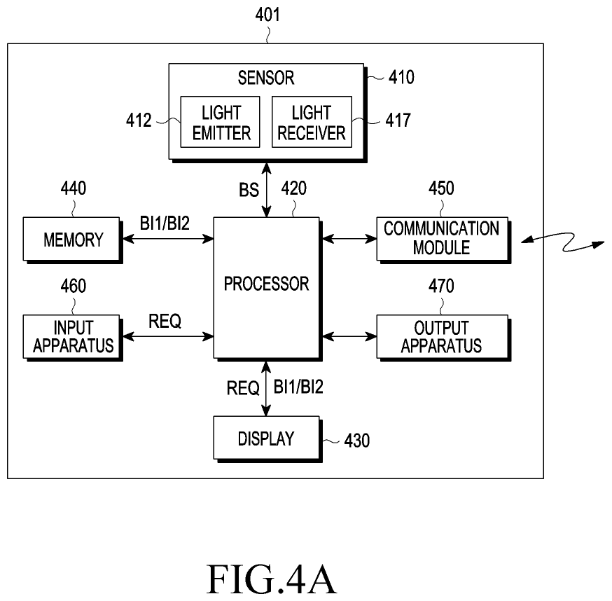

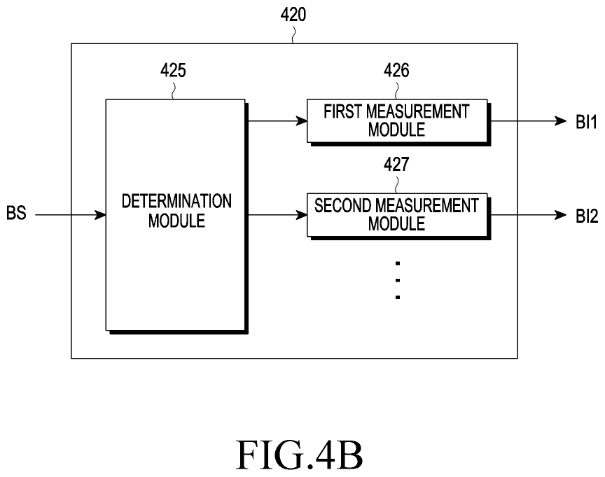

FIG. 4A is a rough block diagram of an electronic device according to various embodiments of the disclosure.

Referring to FIG. 4A, the electronic device 401 (e.g., the electronic device 101 or 201) may include a sensor 410 (e.g., the sensor module 240), a processor 420 (e.g., the processor 120 or 210), a display 430 (e.g., the display 160 or 260), a memory 440 (e.g., the memory 130 or 230), a communication module 450 (e.g., the communication module 220), an input apparatus 460 (e.g., the input apparatus 250), and an output apparatus 470.

The electronic device 401 may be configured to be substantially identical to, or similarly to, the electronic device 101 or 201 described with reference to FIGS. 1 and 2.

The sensor 410 may acquire a biometric signal BS of a user. For example, the sensor 410 may be implemented by a Photoplethysmogram (PPG) sensor.

The sensor 410 may include a light emitter 412 and a light receiver 417. For example, the light emitter 412 may output light (or a light signal) to a user's skin. For example, the light emitter 412 may output at least one of an infrared ray, red light, green light, and/or blue light (or light signal). Further, the light emitter 412 may include at least one of an infrared ray output module, a red light output module, a green light output module, and/or a blue light output module.

The light receiver 417 may receive at least some rays of light (or light signals) reflected from the user's skin (or the user's skin tissue) among rays of light (or light signals) output from the light emitter 412. Further, the light receiver 417 may output a biometric signal BS corresponding to the received light. For example, the light receiver 417 may include a photo diode.

A biometric signal BS may refer to a signal reflected from the user's skin (or the user's skin tissue) among rays of light (or light signals) output from the sensor 410. For example, a biometric signal BS may refer to a signal which is reflected from the user's skin (or the user's skin tissue) and is received through the light receiver 417 of the sensor 410. For example, a sensor signal BS may include a PPG signal.

The processor 420 may control an overall operation of the electronic device 401.

According to an embodiment, the processor 420 may receive a request signal REQ for measurement of a biometric signal. The processor 420 may acquire a biometric signal BS through the sensor 410 in response to the request signal REQ. The processor 420 may measure biometric information of the user on the basis of the biometric signal BS. Further, the processor 420 may provide the user with the measured biometric information through the display 430.

For example, biometric information may include information related to the user's living body, including a heart rate, an oxygen saturation, a blood pressure value, a stress index, and/or the like.

The processor 420 may measure multiple pieces of biometric information of the user by using one biometric signal BS acquired by the sensor 410. For example, the processor 420 may measure multiple pieces of biometric information on the basis of one biometric signal BS during a particular period of time.

According to an embodiment, the processor 420 may measure first biometric information BI1 and second biometric information BI2 on the basis of a biometric signal BS. For example, the processor 420 may determine the second biometric information BI2 to be measured simultaneously with the first biometric information BI1, on the basis of at least one attribute related to measurement of the first biometric information BI1.

For example, the first biometric information BI1 and the second biometric information BI2 may include information related to the user's living body, including a heart rate, an oxygen saturation, a blood pressure value, a stress index, and the like. Further, the first biometric information BI1 and the second biometric information BI2 may be different pieces of biometric information.

Although, for convenience of description, FIG. 4 is illustrated as a configuration in which the processor 420 measures the first biometric information BI1 and the second biometric information BI2, the number of pieces of biometric information measured by the processor 420 is not limited thereto. For example, the processor 420 may measure the first biometric information BI1 and the at least one piece of second biometric information BI2.

For example, an attribute related to measurement of the first biometric information BI may include information on a measurement time, a measurement frequency, and/or measurement light (or a measurement light signal) for the first biometric information BI.

According to an embodiment, the processor 420 may determine the second biometric information BI2 on the basis of a measurement time for the first biometric information BI1. For example, the processor 420 may determine, as the second biometric information BI2, biometric information which can be measured for a time shorter than or equal to the measurement time for the first biometric information BI1.

Hereinafter, for convenience of description, the second biometric information BI2 may refer to biometric information, a measurement time for which is shorter than that of the first biometric information BI1. However, the technical idea of the disclosure is not limited thereto.

According to an embodiment, the processor 420 may determine the second biometric information BI2 on the basis of a frequency band of a biometric signal BS for measurement of the first biometric information BI1. For example, the processor 420 may determine, as the second biometric information BI2, biometric information measured in a frequency band identical to (or different from) a frequency band of a biometric signal BS for measurement of the first biometric information BI1.

According to an embodiment, the processor 420 may determine the second biometric information BI2 on the basis of light (or a light signal) for measurement of the first biometric information BI1. For example, the processor 420 may determine, as the second biometric information BI2, biometric information measured using light (or a light signal) identical to light for measurement of the first biometric information BI1.

The display 430 may display biometric information measured by the processor 420. For example, the display 430 may be implemented by a touch screen.

The display 430 (e.g., a touch screen) may receive an input for measurement of biometric information. Further, the display 430 (e.g., a touch screen) may transmit, to the processor 420, a request signal REQ corresponding to the received input.

The memory 440 may store the biometric information measured by the processor 420. The memory 440 may store the biometric signal BS acquired by the sensor 410. For example, the memory 440 may be implemented by a non-volatile memory or a volatile memory.

According to an embodiment, the memory 440 may store the first biometric information BI1 and the second biometric information BI2.

The communication module 450 may transmit the biometric information, measured by the processor 420, to an external electronic device (not illustrated). Further, the communication module 450 may receive the biometric information measured by an external electronic device.

The input apparatus 460 may receive an input for measurement of biometric information. Further, the input apparatus 460 may transmit, to the processor 420, a request signal REQ corresponding to the received input.

For example, the input apparatus 460 may be implemented by an input means for measuring biometric information. Further, the input apparatus 460 may be implemented by a touch screen of the display 430.

The output apparatus 470 may notify a user of a measurement state of biometric information. For example, the output apparatus 470 may notify the user of a measurement state of biometric information through an auditory means, a tactile means, and a visual means.

FIG. 4B is a block diagram for explaining an operation of the processor illustrated in FIG. 4A.

Referring to FIG. 4B, the processor 420 may include a determination module 425, a first measurement module 426, and a second measurement module 427.

The determination module 425 may determine second biometric information BI2 to be measured in a measurement time for the first biometric information BI1. The determination module 425 may acquire a biometric signal BS from the sensor 410, and may transmit the biometric signal BS to the first measurement module 426 and the second measurement module 427.

The first measurement module 426 may measure first biometric information BI1 on the basis of a first portion of the biometric signal BS. The first measurement module 426 may display the measured first biometric information BI1 on the display 430.

The second measurement module 427 may measure second biometric information BI2 on the basis of a second portion of the biometric signal BS. The second measurement module 427 may display the measured second biometric information BI2 on the display 430.

For example, each of the first portion and the second portion may refer to a part corresponding to a particular time and/or a particular frequency band in the biometric signal BS. The first portion and the second portion may overlap or may be different.

According to an embodiment, when measurement of second biometric information BI2 is completed, the second measurement module 427 may stop the measurement of the second biometric information BI2. Further, the second measurement module 427 may transmit the measured second biometric information BI2 to the first measurement module 426. In this example, the first measurement module 426 may also measure first biometric information BI1 by using the measured second biometric information BI2.

According to another embodiment, even when measurement of second biometric information BI2 is completed, the second measurement module 427 may continuously measure the second biometric information BI2. For example, the second measurement module 427 may measure second biometric information BI2 until measurement of first biometric information BI1 is completed.

Although, for convenience of description, FIG. 4B illustrates only the first measurement module 426 and the second measurement module 427, the number and types of measurement modules, which measure multiple pieces of biometric information, are not limited thereto.

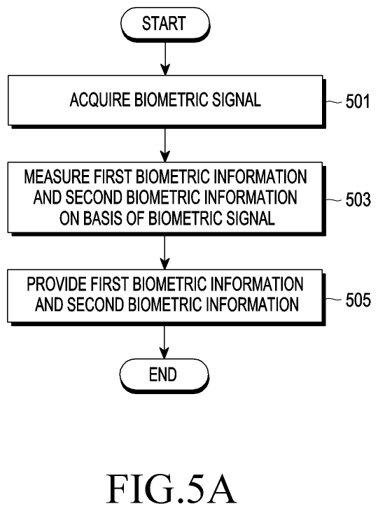

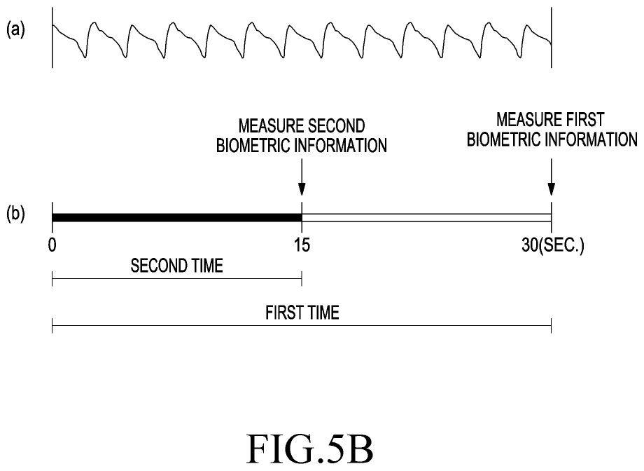

FIG. 5A is a flowchart for explaining an operation of an electronic device according to various embodiments of the disclosure.

Referring to FIG. 5A, a processor (the processor 420 of FIG. 4A) may receive a request signal REQ for requesting measurement of at least one piece of biometric information. For example, the processor 420 may receive a request signal REQ through the display 430 and/or the input apparatus 460. Further, the processor 420 may receive a request signal REQ for requesting measurement of biometric information through the communication module 450 from an external electronic device (e.g., a smart phone or a wearable device).

In operation 501, the processor 420 may acquire a biometric signal BS through the sensor 410. For example, when a user's body part comes in contact with the sensor 410, the processor 420 may acquire a biometric signal BS of the user through the sensor 410.

The processor 420 may measure at least one piece of biometric information on the basis of the biometric signal BS. For example, at least one piece of biometric information measured by the processor 420 may be determined by the user or automatically.

According to an embodiment, in operation 503, the processor 420 may measure first biometric information BI1 and second biometric information BI2 on the basis of the biometric signal BS. For example, the processor 420 may measure first biometric information BI1 on the basis of a first portion (e.g., a first time and/or a first frequency band) of the biometric signal BS, and may measure second biometric information BI2 on the basis of a second portion (e.g., a second time and/or a second frequency band) of the biometric signal BS.

In operation 505, the processor 420 may provide the measured first biometric information BI1 and the measured second biometric information BI2 through the display 430.

FIG. 5B is a graph for explaining the operation of the electronic device of FIG. 5A.

Referring to FIG. 5B, the processor 420 may measure first biometric information BI1 on the basis of a first portion of a biometric signal BS, and may measure second biometric information BI2 on the basis of a second portion of the biometric signal BS.

Referring to (a) of FIG. 5B, the processor 420 may receive a biometric signal BS by using the sensor 410.

Referring to (b) of FIG. 5B, the processor 420 may measure first biometric information BI1 on the basis of a biometric signal BS corresponding to a first time. Further, the processor 420 may measure second biometric information BI2 on the basis of a biometric signal BS corresponding to a second time. For example, the second time may refer to at least a partial time included in the first time.

According to an embodiment, the processor 420 may measure first biometric information BI1 on the basis of a biometric signal BS corresponding to a first time (e.g., 0 to 30 seconds). Further, the processor 420 may measure second biometric information BI2 on the basis of a biometric signal BS corresponding to a second time (e.g., 0 to 15 seconds). That is, the processor 420 may measure the first biometric information BI1 and the second biometric information BI2 for the first time (e.g., 0 to 30 seconds).

According to another embodiment, the processor 420 may measure first biometric information BI1 on the basis of a biometric signal BS corresponding to a first time (e.g., 0 to 30 seconds) and may measure second biometric information BI2 on the basis of a biometric signal BS corresponding to a third time (e.g., 15 to 30 seconds).

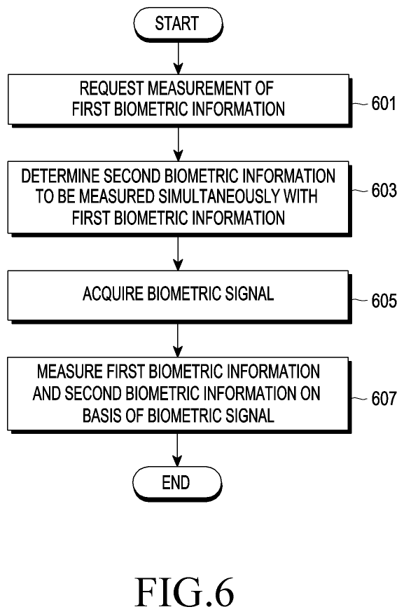

FIG. 6 is a flowchart for explaining an operation of an electronic device according to various embodiments of the disclosure.

Referring to FIG. 6, in operation 601, a processor (the processor 420 of FIG. 4A) may receive a request signal REQ for requesting measurement of at least one piece of biometric information. For example, the processor 420 may receive a request signal REQ through the display 430 and/or the input apparatus 460.

In operation 603, the processor 420 may determine second biometric information BI2 to be measured simultaneously with the first biometric information BI1. For example, the processor 420 may determine second biometric information BI2 to be measured together with the first biometric information BI1 during measurement of the first biometric information BI1. For example, second biometric information BI2 may be determined by a user or automatically.

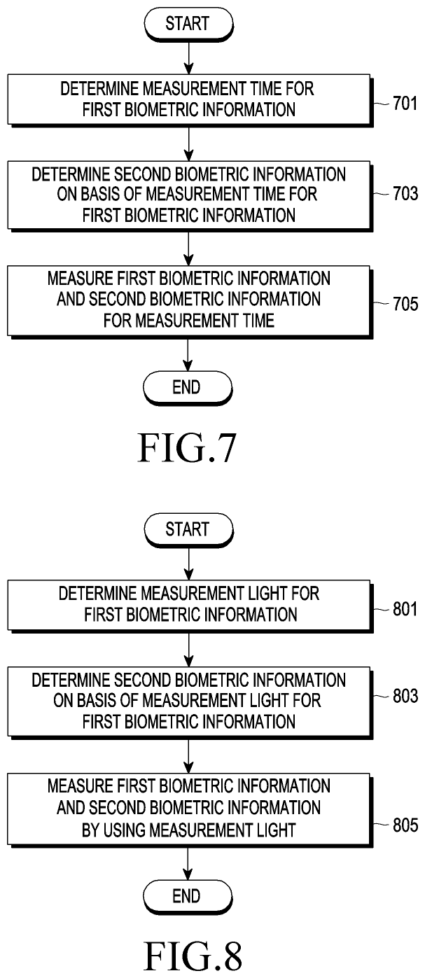

Meanwhile, embodiments in which the processor 420 determines second biometric information BI2 to be measured simultaneously with first biometric information BI1 will be described in more detail below with reference to FIGS. 7 to 9.

In operation 605, the processor 420 may acquire a biometric signal BS through the sensor 410. For example, when the user's body part comes in contact with the sensor 410, the processor 420 may acquire a biometric signal BS of the user through the sensor 410.

In operation 607, the processor 420 may measure the first biometric information BI1 and the second biometric information BI2 on the basis of the biometric signal BS. For example, the processor 420 may measure the first biometric information BI1 on the basis of a first portion (e.g., a first time and/or a first frequency band) of the biometric signal BS, and may measure the second biometric information BI2 on the basis of a second portion (e.g., a second time and/or a second frequency band) of the biometric signal BS.

FIG. 7 is a flowchart for explaining an operation of an electronic device according to various embodiments of the disclosure.

Referring to FIG. 7, a processor (the processor 420 of FIG. 4) may determine second biometric information BI2 to be measured simultaneously with first biometric information BI1. For example, the processor 420 may determine second biometric information BI2 to be measured together with first biometric information BI1 during measurement of the first biometric information BI1.

In operation 701, the processor 420 may determine a measurement time for the first biometric information BI1. For example, the processor 420 may determine a time, for which a biometric signal BS is acquired using the sensor 410 in order to measure the first biometric information BI1, as a measurement time for the first biometric information BI1.

In operation 703, the processor 420 may determine second biometric information BI2 on the basis of the measurement time for the first biometric information BI1. For example, second biometric information BI2 may refer to biometric information which can be measured in the measurement time for the first biometric information BI1 among multiple pieces of biometric information of the user.

When second biometric information BI2 is determined, the processor 420 may acquire a biometric signal BS by using the sensor 410.

In operation 705, the processor 420 may measure the first biometric information BI1 and the second biometric information BI2 for the measurement time for the first biometric information BI1. For example, the processor 420 may acquire a biometric signal BS through the sensor 410, may acquire the first biometric information BI1 on the basis of at least a part of a biometric signal BS acquired for a first time, and may acquire the second biometric information BI2 on the basis of at least a part of a biometric signal BS acquired for a second time. In this example, the second time may be shorter than or equal to the first time.

FIG. 8 is a flowchart for explaining an operation of an electronic device according to various embodiments of the disclosure.

Referring to FIG. 8, a processor (the processor 420 of FIG. 4) may determine second biometric information BI2 to be measured simultaneously with first biometric information BI1. For example, the processor 420 may determine second biometric information BI2 to be measured together with first biometric information BI1 during measurement of the first biometric information BI1.

In operation 801, the processor 420 may determine measurement light for the first biometric information BI1. For example, the processor 420 may determine light (or the type of light) output from the sensor 410 in order to measure first biometric information BI1, as measurement light for the first biometric information BI1.

In operation 803, the processor 420 may determine second biometric information BI2 on the basis of the measurement light (or the type of light) for the first biometric information BI1. For example, second biometric information BI2 may refer to biometric information which can be measured using light identical to the measurement light for the first biometric information BI1 among multiple pieces of biometric information of a user. For example, when measurement light for the first biometric information BI1 is infrared light, the processor 420 may determine biometric information, which can be measured using infrared light, as second biometric information BI2.

When the second biometric information BI2 is determined, the processor 420 may acquire a biometric signal BS by using the sensor 410. For example, when measurement light for the first biometric information BI1 is infrared light, the processor 420 may acquire a biometric signal BS corresponding to infrared light by using the sensor 410.

In operation 805, the processor 420 may measure the first biometric information BI1 and the second biometric information BI2 by using the measurement light for the first biometric information BI1. For example, when measurement light for the first biometric information BI1 is infrared light, the processor 420 may measure the first biometric information BI1 and the second biometric information BI2 on the basis of a biometric signal BS corresponding to infrared light.

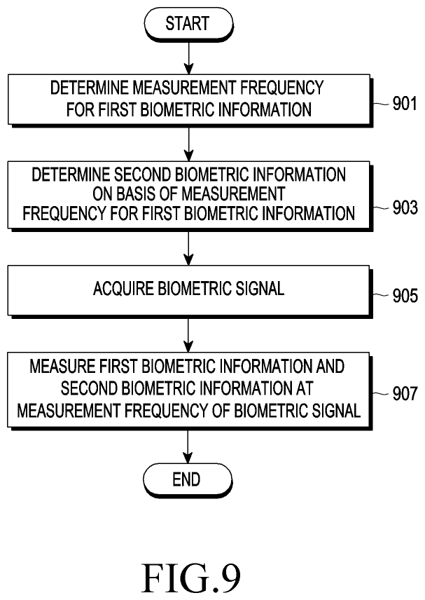

FIG. 9 is a flowchart for explaining an operation of an electronic device according to various embodiments of the disclosure.

Referring to FIG. 9, a processor (the processor 420 of FIG. 4) may determine second biometric information BI2 to be measured simultaneously with first biometric information BI1. For example, the processor 420 may determine second biometric information BI2 to be measured together with first biometric information BI1 during measurement of the first biometric information BI1.

In operation 901, the processor 420 may determine a measurement frequency for the first biometric information BI1. For example, the processor 420 may determine a frequency band of a biometric signal BS for measure of first biometric information BI1, as a measurement frequency for the first biometric information BI1.

In operation 903, the processor 420 may determine second biometric information BI2 on the basis of the measurement frequency for the first biometric information BI1. For example, second biometric information BI2 may refer to biometric information which can be measured at the measurement frequency for the first biometric information BI1 among multiple pieces of biometric information of a user. Further, the second biometric information BI2 may refer to biometric information which can be measured at a measurement frequency different from the measurement frequency for the first biometric information BI1 among the multiple pieces of biometric information of the user.

In operation 905, when second biometric information BI2 is determined, the processor 420 may acquire a biometric signal BS by using the sensor 410.

In operation 907, the processor 420 may measure the first biometric information BI1 and the second biometric information BI2 at the measurement frequency for the first biometric information BI1.

According to an embodiment, the processor 420 may measure first biometric information BI1 and second biometric information BI2 at a measurement frequency for the first biometric information BI1 in a biometric signal BS.

According to another embodiment, the processor 420 may measure first biometric information BI1 at a first measurement frequency in a biometric signal BS, and may measure second biometric information BI2 at a second measurement frequency in the biometric signal BS. For example, the second measurement frequency may be different from the first measurement frequency. In this example, the processor 420 may separate frequencies respectively corresponding to bands by using a filter.

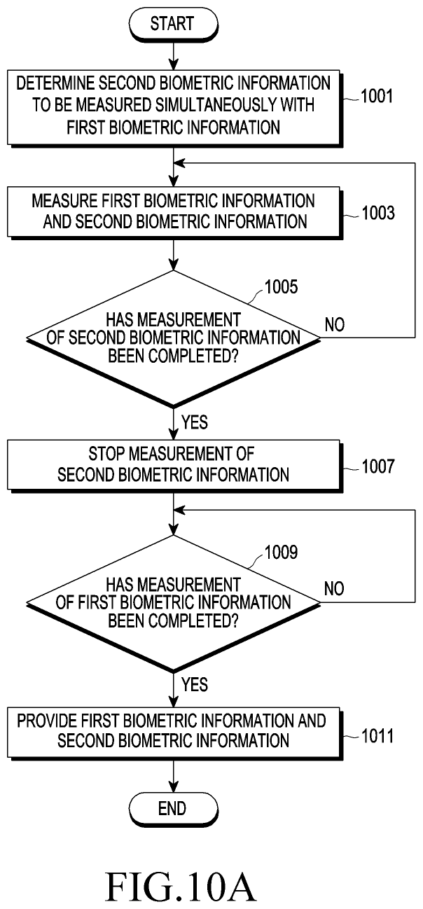

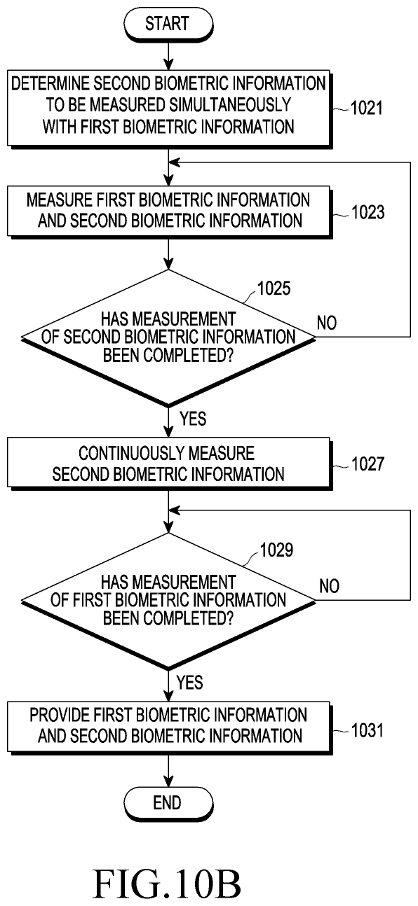

FIG. 10A is a flowchart for explaining an operation of an electronic device according to various embodiments of the disclosure.

Referring to FIG. 10A, a processor (the processor 420 of FIG. 4A) may receive a request signal REQ for requesting measurement of at least one piece of biometric information. The processor 420 may measure biometric information of a user in response to the request signal REQ.

In operation 1001, the processor 420 may determine second biometric information BI2 to be measured simultaneously with first biometric information BI1. For example, the processor 420 may determine second biometric information BI2 to be measured together with first biometric information BI1 during measurement of the first biometric information BI1. For example, a measurement time for the second biometric information BI2 may be shorter than or equal to a measurement time for the first biometric information BI1.

The processor 420 may acquire a biometric signal BS through the sensor 410. The processor 420 may measure the first biometric information BI1 and the second biometric information BI2 on the basis of the biometric signal BS.

In operation 1005, the processor 420 may determine whether measurement of the second biometric information BI2 has been completed.

According to an embodiment, when the measurement of the second biometric information BI2 has been completed (Yes in operation 1005), in operation 1007, the processor 420 may stop the measurement of the second biometric information BI2. For example, the processor 420 may stop an operation of a second measurement module (the second measurement module 427 of FIG. 4B). Meanwhile, when the measurement of the second biometric information BI2 has not been completed, the processor 420 may continuously measure the second biometric information BI2.

According to an embodiment, when the measurement of the second biometric information BI2 has been completed (Yes in operation 1005), the processor 420 may provide the measured second biometric information BI2 through the display 430. For example, the processor 420 may stop the measurement of the second biometric information BI2, and may display the measured second biometric information BI2 on the display 430. That is, when the measurement of the second biometric information BI2 has been completed, the processor 420 may first provide the second biometric information BI2, the measurement of which has been completed.

In operation 1009, the processor 420 may determine whether the measurement of the first biometric information BI1 has been completed.

When the measurement of the first biometric information BI1 has not been completed (No in operation 1009), the processor 420 may continuously measure the first biometric information BI1.

When the measurement of the second biometric information BI2 has been completed (Yes in operation 1009), in operation 1011, the processor 420 may provide the first biometric information BI1 and the second biometric information BI2 to the user through the display 430.

FIG. 10B is a flowchart for explaining an operation of an electronic device according to various embodiments of the disclosure.

Referring to FIG. 10B, operations 1021 to 1031 except for operation 1027 in FIG. 10B may be configured to be substantially identical to, or similarly to, operations 1001 to 1011 described with reference to FIG. 10A.

In operation 1021, a processor (the processor 420 of FIG. 4) may determine second biometric information BI2 to be measured simultaneously with first biometric information BI1, in response to a request for measurement of biometric information.

In operation 1023, the processor 420 may measure the first biometric information BI1 and the second biometric information BI2 on the basis of a biometric signal BS acquired through the sensor 410.

In operation 1025, the processor 420 may determine whether measurement of the second biometric information BI2 has been completed.

According to an embodiment, even when the measurement of the second biometric information BI2 has been completed (Yes in operation 1025), in operation 1027, the processor 420 may continuously measure the second biometric information BI2. For example, the processor 420 may continuously measure the second biometric information BI2, and thus can increase the reliability of the second biometric information BI2.

In operation 1029, the processor 420 may determine whether the measurement of the first biometric information BI1 has been completed.

When the measurement of the first biometric information BI1 has not been completed (No in operation 1029), the processor 420 may continuously measure the first biometric information BI1.

When the measurement of the second biometric information BI2 has been completed (Yes in operation 1029), in operation 1031, the processor 420 may provide the first biometric information BI1 and the second biometric information BI2 to a user through the display 430.

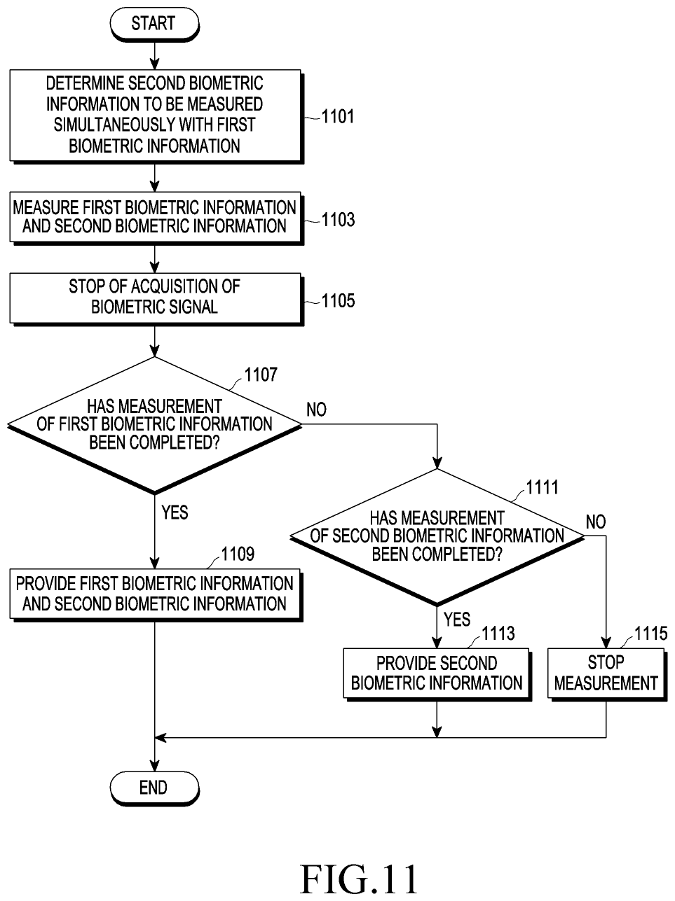

FIG. 11 is a flowchart for explaining an operation of an electronic device according to various embodiments of the disclosure.

Referring to FIG. 11, a processor (the processor 420 of FIG. 4A) may receive a request signal REQ for requesting measurement of at least one piece of biometric information. The processor 420 may measure biometric information of a user in response to the request signal REQ.

In operation 1101, the processor 420 may determine second biometric information BI2 to be measured simultaneously with first biometric information BI1, in response to the request signal REQ.

The processor 420 may acquire a biometric signal BS of the user by using the sensor 410.

In operation 1103, the processor 420 may measure the first biometric information BI1 and the second biometric information BI2 on the basis of the biometric signal BS acquired through the sensor 410.

In operation 1105, acquisition of a biometric signal BS from the sensor 410 may be stopped. For example, when a user's body part does not come in contact with the sensor 410, acquisition of a biometric signal BS may be stopped.

In operation 1107, when the acquisition of the biometric signal BS is stopped, the processor 420 may determine whether measurement of the first biometric information BI1 has been completed. For example, when a measurement time for the second biometric information BI2 is shorter than or equal to a measurement time for the first biometric information BI1, if the measurement of the first biometric information BI1 is completed, the measurement of the second biometric information BI2 may also be completed.

When the measurement of the first biometric information BI1 has been completed (Yes in operation 1107), in operation 1109, the processor 420 may provide the measured first biometric information BI1 and the measured second biometric information BI2 through a display (the display 430 of FIG. 4).

When the measurement of the first biometric information BI1 has not been completed (No in operation 1107), in operation 1111, the processor 420 may determine whether the measurement of the second biometric information BI2 has been completed. For example, if a measurement time for the second biometric information BI2 is shorter than or equal to a measurement time for the first biometric information BI1, even when measurement of the first biometric information BI1 is not completed, measurement of the second biometric information BI2 may be completed.

When the measurement of the second biometric information BI2 has been completed (Yes in operation 1111), in operation 1113, the processor 420 may provide the measured second biometric information BI2 through the display 430.

When the measurement of the second biometric information BI2 has not been completed (No in operation 1111), in operation 1115, the processor 420 may stop the measurement of the first biometric information BI1 and the measurement of the second biometric information BI2. For example, since the measurement of the first biometric information BI1 and the measurement of the second biometric information BI2 have not been completed, the processor 420 may not provide the first biometric information BI1 and the second biometric information BI2. In this example, the processor 420 may display, on the display 430, information indicating a failure in measurement of biometric information.

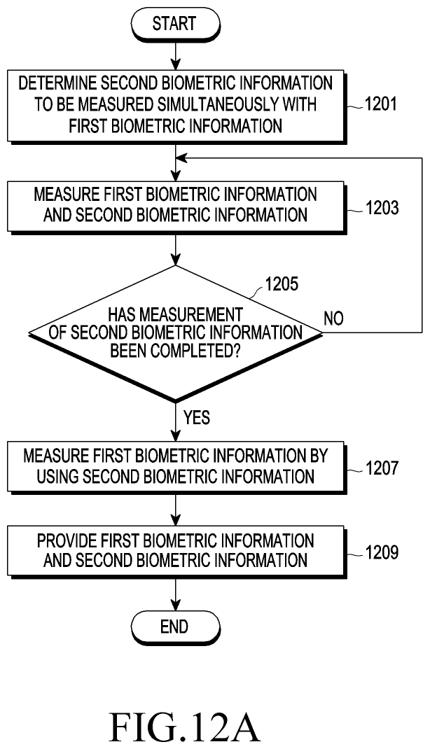

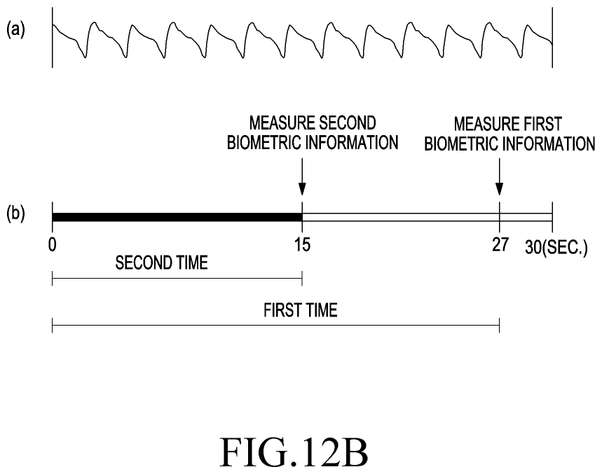

FIG. 12A is a flowchart for explaining an operation of an electronic device according to various embodiments of the disclosure.

Referring to FIG. 12A, a processor (the processor 420 of FIG. 4A) may receive a request signal REQ for requesting measurement of at least one piece of biometric information. The processor 420 may measure biometric information of a user in response to the request signal REQ.

In operation 1201, the processor 420 may determine second biometric information BI2 to be measured simultaneously with first biometric information BI1, in response to the request signal REQ.

The processor 420 may acquire a biometric signal BS of a user by using the sensor 410.

In operation 1203, the processor 420 may measure the first biometric information BI1 and the second biometric information BI2 on the basis of the biometric signal BS.

In operation 1205, the processor 420 may determine whether measurement of the second biometric information BI2 has been completed. For example, if a sensing time necessary to measure the second biometric information BI2 is shorter than or equal to a sensing time necessary to measure the first biometric information BI1, even when measurement of the first biometric information BI1 is not completed, measurement of the second biometric information BI2 may be completed.

When the measurement of the second biometric information BI2 has not been completed (No in operation 1205), the processor 420 may continuously measure the second biometric information BI2.

When the measurement of the second biometric information BI2 has been completed (Yes in operation 1205), in operation 1207, the processor 420 may measure the first biometric information BI1 by using the measured second biometric information BI2. For example, the processor 420 may reduce a measurement time for the first biometric information BI1 by using the measured second biometric information BI2. Further, the processor 420 may increase the reliability of the first biometric information BI1 by using the measured second biometric information BI2. In this example, the second biometric information BI2 may correlate with the first biometric information BI1.

In operation 1209, the processor 420 may provide the measured first biometric information BI1 and the measured second biometric information BI2 through a display (the display 430 of FIG. 4).

FIG. 12B is a graph for explaining the operation of the electronic device of FIG. 12A.

Referring to FIG. 12B, the processor 420 may measure first biometric information BI1 on the basis of a first portion of a biometric signal BS, and may measure second biometric information BI2 on the basis of a second portion of the biometric signal BS.

Referring to (a) of FIG. 12B, the processor 420 may receive a biometric signal BS by using the sensor 410.

Referring to (b) of FIG. 12B, the processor 420 may measure second biometric information BI2 on the basis of a biometric signal BS corresponding to a second time (e.g., 0 to 15 seconds). Further, the processor 420 may measure first biometric information BI1 on the basis of a biometric signal BS corresponding to a first time (e.g., 0 to 27 seconds) by using the measured second biometric information BI2.

That is, when the second biometric information BI2 correlates with the first biometric information, the processor 420 may reduce a measurement time for the first biometric information BI1 by using the measured second biometric information BI2. For example, the processor 420 may measure the first biometric information BI1 and the second biometric information BI2, for a time (e.g., 0 to 27 seconds) shorter than the measurement for the first biometric information BI1 illustrated in (b) of FIG. 5B.

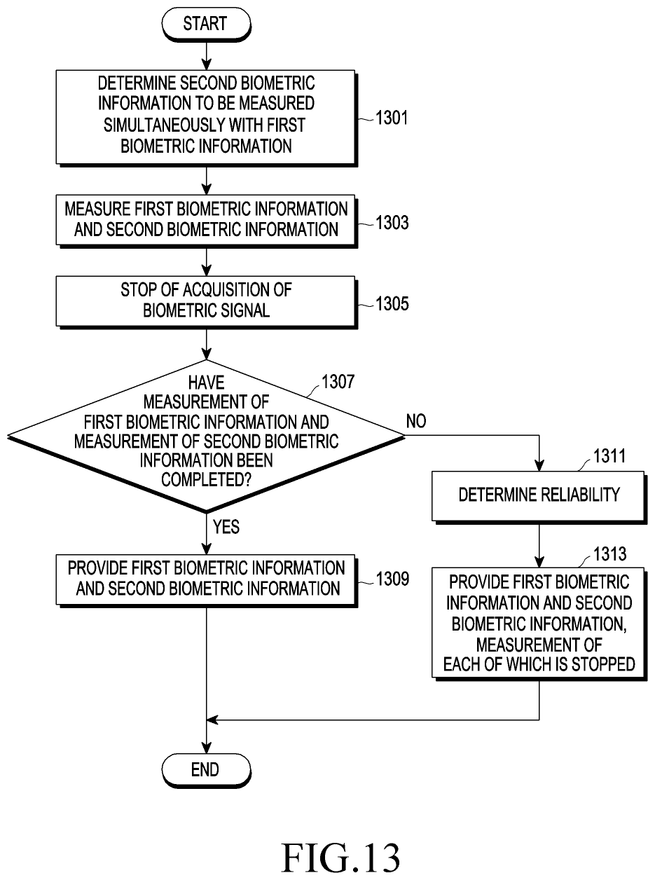

FIG. 13 is a flowchart for explaining an operation of an electronic device according to various embodiments of the disclosure.

Referring to FIG. 13, a processor (the processor 420 of FIG. 4A) may receive a request signal REQ for requesting measurement of at least one piece of biometric information. The processor 420 may measure biometric information of a user in response to the request signal REQ.

In operation 1301, the processor 420 may determine second biometric information BI2 to be measured simultaneously with first biometric information BI1, in response to the request signal REQ.

The processor 420 may acquire a biometric signal BS of the user by using the sensor 410.

In operation 1303, the processor 420 may measure the first biometric information BI1 and the second biometric information BI2 on the basis of the biometric signal BS.

In operation 1305, acquisition of a biometric signal BS from the sensor 410 may be stopped. For example, when the user's body part does not come in contact with the sensor 410, acquisition of a biometric signal BS may be stopped.

When the acquisition of the biometric signal BS is stopped, in operation 1307, the processor 420 may determine whether measurement of the first biometric information BI1 and measurement of the second biometric information BI2 have been completed.

When the measurement of the first biometric information BI1 and the measurement of the second biometric information BI2 have been completed (Yes in operation 1307), in operation 1309, the processor 420 may provide the measured first biometric information BI1 and the measured second biometric information BI2 through a display (the display 430 of FIG. 4).

When the measurement of the first biometric information BI1 and the measurement of the second biometric information BI2 have not been completed (No in operation 1307), in operation 1311, the processor 420 may determine the reliability of the first biometric information BI1 and the second biometric information BI2, the measurement of each of which has not been completed.

The processor 420 may determine the reliability of the first biometric information BI1 and the second biometric information BI2, the measurement of each of which has not been completed, on the basis of a time necessary to measure the first biometric information BI1 and a time necessary to measure the second biometric information BI2. For example, when a time necessary to measure the first biometric information BI1 is 30 seconds, the processor 420 may determine, as 33%, the reliability of the first biometric information BI1 which is based on a biometric signal BS acquired for 10 seconds.

In operation 1313, the processor 420 may provide the first biometric information BI1 and the second biometric information BI2, the measurement of each of which has not been completed, through the display 430. In this example, the processor 420 may provide, through the display 430, information indicating that the measurement has not been completed and the reliability of the first biometric information BI1 and the second biometric information BI2.

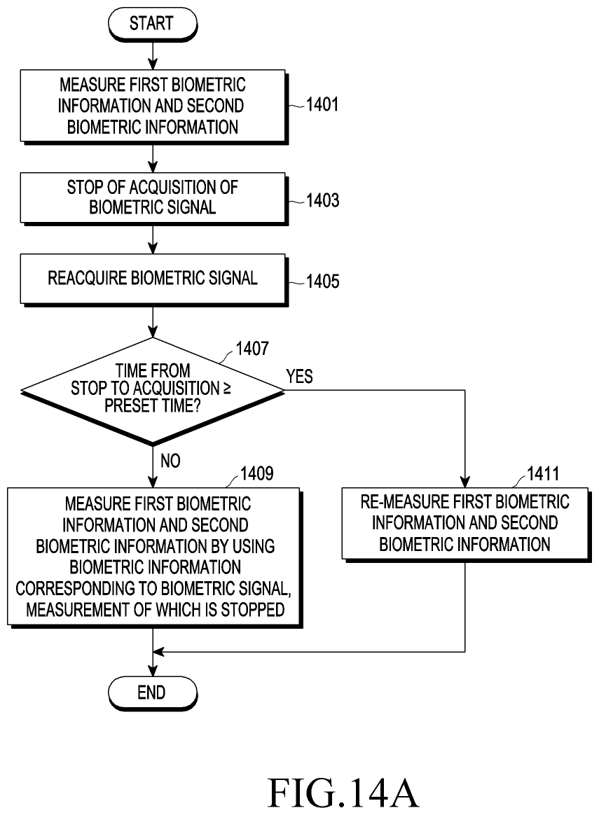

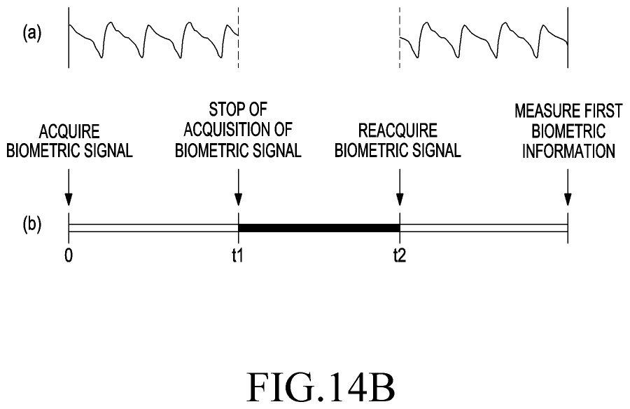

FIG. 14A is a flowchart for explaining an operation of an electronic device according to various embodiments of the disclosure.

Referring to FIG. 14A, in operation 1401, a processor (the processor 420 of FIG. 4) may measure first biometric information BI1 and second biometric information BI2 on the basis of a biometric signal BS.

In operation 1403, acquisition of a biometric signal BS from the sensor 410 may be stopped. For example, when a user's body part does not come in contact with the sensor 410, acquisition of a biometric signal BS may be stopped. The processor 420 may store, in the memory 440, a biometric signal BS until the acquisition thereof is stopped. Further, the processor 420 may store, in the memory 440, biometric information measured on the basis of the biometric signal BS until the acquisition thereof is stopped.

After the acquisition of the biometric signal BS is stopped, in operation 1405, the processor 420 may reacquire a biometric signal BS from the sensor 410. For example, after a user's body part does not come in contact with the sensor 410, when the user's body part again comes in contact with the sensor 410, the processor 420 may resume acquisition of a biometric signal BS.

In operation 1407, the processor 420 may compare, with a preset time, a time (hereinafter “reacquisition time”) between a time point at which acquisition of a biometric signal is stopped, and a time point at which acquisition thereof is resumed. For example, the preset time may refer to a time for which biometric information measured until a time point, at which the acquisition of the biometric signal is stopped, has a reliability higher than or equal to a predetermined level with reference to a reacquisition time.

When a reacquisition time is shorter than the preset time (No in operation 1407), the processor 420 may measure first biometric information BI1 and second biometric information BI2 by using a biometric signal acquired until the acquisition of the biometric signal is stopped. For example, in operation 1409, the processor 420 may measure first biometric information BI1 and second biometric information BI2 by using a biometric signal BS acquired until the acquisition of the biometric signal is stopped (or biometric information measured on the basis of the acquired biometric signal) and the reacquired biometric signal BS.

For example, when first biometric information BI1, for which a sensing time requires a total of 30 seconds, is measured, the processor 420 may measure the first biometric information BI1 by using a biometric signal, acquired for 10 seconds until sensing is stopped and a biometric signal BS acquired for 20 seconds from a time point at which sensing is resumed.

When a reacquisition time is longer than or equal to the preset time (Yes in operation 1407), in operation 1411, the processor 420 may not use a biometric signal acquired until sensing is stopped. That is, in operation 1411, the processor 420 may re-measure first biometric information BI1 and second biometric information BI2 on the basis of the reacquired biometric signal BS.

For example, when first biometric information BI1, for which a sensing time requires a total of 30 seconds, is measured, the processor 420 may not use a biometric signal, acquired for 10 seconds until sensing is stopped and may measure the first biometric information BI1 by using a biometric signal BS acquired for 30 seconds from a time point at which sensing is resumed.

FIG. 14B is a graph for explaining the operation of the electronic device of FIG. 14A.

Referring to FIG. 14B, after acquisition of a biometric signal BS is stopped, when acquisition thereof is resumed, the processor 420 may measure first biometric information BI1 and second biometric information BI2 by using biometric information measured until the acquisition of the biometric signal BS is stopped, according to a reacquisition time.

Referring to (a) of FIG. 14B, the processor 420 may acquire a biometric signal BS by using the sensor 410. For example, when a user's body part does not come in contact with the sensor 410, acquisition of the biometric signal BS may be stopped. Further, when the user's body part again comes in contact with the sensor 410, acquisition of the biometric signal BS may be resumed.

Referring to (b) of FIG. 14B, acquisition of a biometric signal BS may be stopped at a first time point (e.g., t1 seconds). Further, acquisition of the biometric signal BS may be resumed at a second time point (e.g., t2 seconds).

The processor 420 may measure first biometric information BI1 by using biometric information measured until acquisition of the biometric signal is stopped, according to a reacquisition time (t2-t1 seconds).

For example, when a reacquisition time (t2-t1 seconds) is shorter than a preset time, the processor 420 may measure first biometric information BI1 by using biometric information measured on the basis of a biometric signal acquired until the acquisition of the biometric signal is stopped. In this example, the processor 420 may reduce a measurement time for the first biometric information BI1 by using the biometric information measured until the acquisition of the biometric signal is stopped.

In contrast, when a reacquisition time (t241 seconds) is longer than or equal to the preset time, the processor 420 may not use a biometric signal acquired until the acquisition of the biometric signal is stopped and may measure first biometric information BI1 on the basis of a biometric signal BS acquired after a second time point (e.g., t2 seconds).

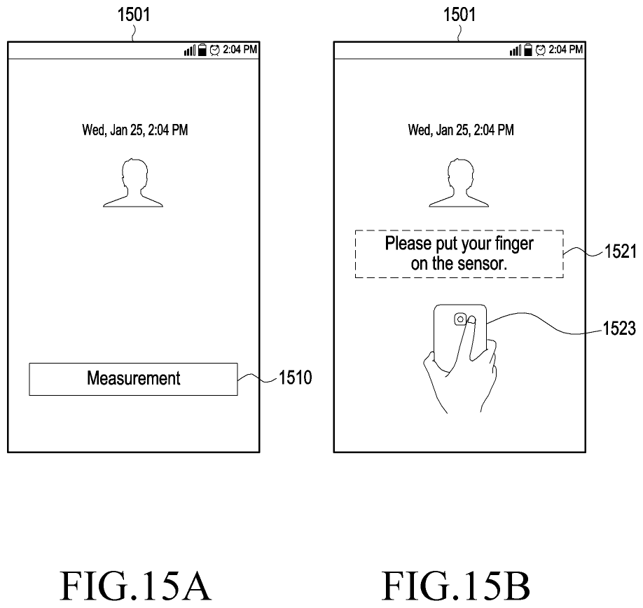

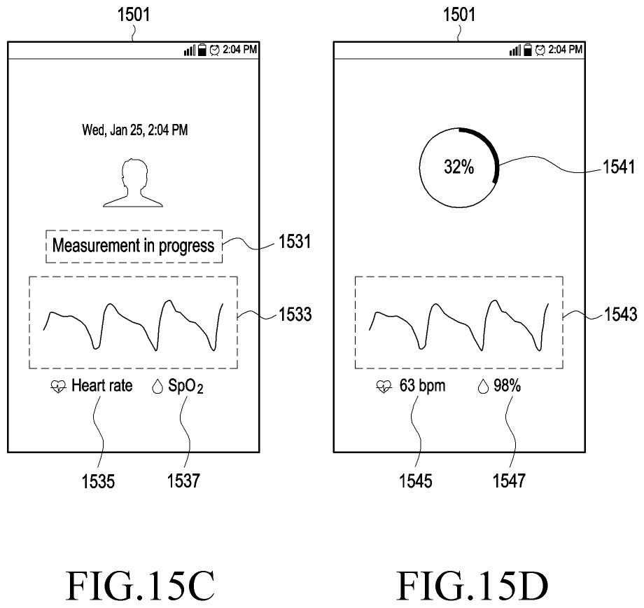

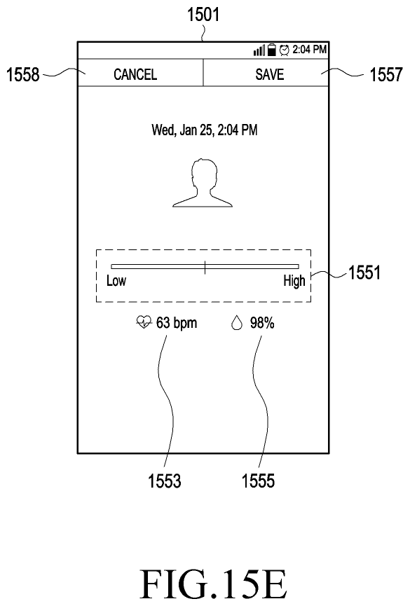

FIGS. 15A, 15B, 15C, 15D, and 15E each illustrate a user interface provided by an electronic device according to various embodiments of the disclosure.

Referring to FIGS. 15A, 15B, 15C, 15D, and 15E, the electronic device 1501 may be configured to be substantially identical to, or similar to, the electronic device 401 described with reference to FIG. 4A.

Referring to FIG. 15A, a processor (the processor 420 of FIG. 4) may execute an application configured to measure biometric information of a user in response to a request signal REQ.

The processor 420 may display a measurement object 1510 for measurement of biometric information of the user. The processor 420 may measure biometric information of the user in response to an input through the measurement object 1510.

Referring to FIG. 15B, the processor 420 may display information for acquisition of a biometric signal BS of the user through a sensor. For example, the processor 420 may display, on the display 430, notification or information for acquisition of a biometric signal BS.

For example, the processor 420 may display text information 1521 representing “Please put your finger on the sensor”. Further, the processor 420 may display image information 1523 representing information on a finger which comes in contact with the sensor.

Meanwhile, the processor 420 may determine at least one piece of biometric information to be measured. For example, the processor 420 may determine first biometric information BI1 and second biometric information BI2, as biometric information to be measured using a sensor. First biometric information BI1 and second biometric information BI2 may be determined by the user, or may be automatically determined by the processor 420.

Referring to FIG. 15C, the processor 420 may display a measurement state of biometric information (e.g., may display “measurement in progress”) through a first guide object 1531. Further, the processor 420 may display a biometric signal (e.g., may display a waveform of a biometric signal) acquired using the sensor through a second guide object 1533.

The processor 420 may display first biometric information 1535 and second biometric information 1537 to be measured using the sensor. For example, the processor 420 may display measurement of a heart rate through the first biometric information 1535 and may display measurement of an oxygen saturation (SpO₂) through the second biometric information 1537.

Referring to FIG. 15D, the processor 420 may display a graph (e.g., may display “32%”) corresponding to a measurement state of biometric information through a third guide object 1541. In this example, the graph may display progress information of measurement by using an image and text. Further, the processor 420 may display a biometric signal (e.g., may display a waveform of a biometric signal) acquired using the sensor through a fourth guide object 1543.

The processor 420 may display first biometric information 1545 and second biometric information 1547 which are measured. For example, the processor 420 may display information (e.g., 63 bpm) on a heart rate 1545 and information (e.g., 98%) on an oxygen saturation 1547 which are measured in real time.

Referring to FIG. 15E, the processor 420 may display a graph representing the user's body state corresponding to the measured biometric information through a fifth guide object 1551. Further, the processor 420 may display first biometric information 1553 and second biometric information 1555, the measurement of each of which has been completed. For example, the processor 420 may display information (e.g., 63 bpm) on a heart rate 1553 and information (e.g., 98%) on an oxygen saturation 1555.

The processor 420 may further display a storage object 1557 and a cancellation object 1558. The processor 420 may store the measured first biometric information 1553 and the measured second biometric information 1555 in the memory 440 in response to an input through the storage object 1557. Further, the processor 420 may not store the measured first biometric information 1553 and the measured second biometric information 1555 in response to an input through the cancellation object 1558.

An electronic device according to various embodiments of the disclosure may include: a sensor configured to acquire a biometric signal; and a processor, wherein the processor is configured to: acquire a biometric signal by using the sensor; measure first biometric information at least on the basis of a first portion of the biometric signal; measure second biometric information at least on the basis of a second portion which is at least a part of the first portion; and display at least one piece of information among the first biometric information and the second biometric information, on a display functionally connected to the electronic device.

The processor may be configured to determine the second biometric information capable of being measured simultaneously with the first biometric information at least on the basis of at least one attribute related to measurement of the first biometric information.

The processor may be configured to verify a measurement time for the measurement of the first biometric information, as at least one element among the at least one attribute.

The processor may be configured to verify a type of light of the sensor configured to measure the first biometric information, as at least one element among the at least one attribute.

The processor may be configured to verify a frequency band of the biometric signal for the measurement of the first biometric information, as at least one element among the at least one attribute.

The processor may be configured to display the second biometric information on the display when measurement of the second biometric information is completed.

The processor may be configured to measure the first biometric information by using the measured second biometric information.

The processor may be configured to: make a determination of whether measurement of the first biometric information has been completed; and continuously measure the second biometric information on the basis of at least a part of the biometric signal according to a result of the determination.

The processor may be configured to: when acquisition of the biometric signal from the sensor is stopped, make a determination of whether measurement of the first biometric information and measurement of the second biometric information have been completed and display the first biometric information or the second biometric information measured until acquisition of the biometric signal is stopped on the display at least on the basis of a result of the determination.

The processor may be configured to: when acquisition of the biometric signal from the sensor is stopped, verify a time until acquisition of another biometric signal through the sensor; when the verified time satisfies a designated condition, measure the first biometric information or the second biometric information by using the biometric signal and another biometric signal; and when the verified time does not satisfy the designated condition, measure the first biometric information or the second biometric information by using another biometric signal.

An operating method of an electronic device according to various embodiments of the disclosure may include: acquiring a biometric signal by using a sensor of the electronic device; measuring first biometric information at least on the basis of a first portion of the biometric signal, and measuring second biometric information at least on the basis of a second portion which is at least a part of the first portion; and displaying at least one piece of information among the first biometric information and the second biometric information, on a display functionally connected to the electronic device.

The measuring of the first biometric information at least on the basis of the first portion of the biometric signal and the measuring of the second biometric information may include determining the second biometric information capable of being measured simultaneously with the first biometric information at least on the basis of at least one attribute related to measurement of the first biometric information.

The determining of the second biometric information may include verifying a measurement time for the measurement of the first biometric information as at least one element among the at least one attribute.

The determining of the second biometric information may include verifying a type of light of the sensor configured to measure the first biometric information, as at least one element among the at least one attribute.

The determining of the second biometric information may include verifying a frequency band of the biometric signal for the measurement of the first biometric information, as at least one element among the at least one attribute.

The operating method of the electronic device may further include displaying the second biometric information on the display when measurement of the second biometric information is completed.

The measuring of the second biometric information may include: verifying whether measurement of the first biometric information has been completed and continuously measuring the second biometric information on the basis of at least a part of the biometric signal.

The displaying of the first biometric information and the second biometric information may include: when acquisition of the biometric signal from the sensor is stopped, making a determination of whether measurement of the first biometric information and measurement of the second biometric information have been completed and displaying the first biometric information or the second biometric information measured until acquisition of the biometric signal is stopped on the display at least on the basis of a result of the determination.

The measuring of the first biometric information and the second biometric information may include: when acquisition of the biometric signal from the sensor is stopped, verifying a time until acquisition of another biometric signal through the sensor; when the verified time satisfies a designated condition, measuring the first biometric information or the second biometric information by using the biometric signal and another biometric signal; and when the verified time does not satisfy the designated condition, measuring the first biometric information or the second biometric information by using another biometric signal.

A computer-readable recording medium according to various embodiments of the disclosure may execute an operating method of an electronic device, the operating method including: acquiring a biometric signal by using a sensor of an electronic device; measuring first biometric information at least on the basis of a first portion of the biometric signal, and measuring second biometric information at least on the basis of a second portion which is at least a part of the first portion; and displaying at least one piece of information among the first biometric information and the second biometric information on a display functionally connected to the electronic device.

Each of the above-described elements of the electronic device may include one or more components, and the name of the corresponding element may vary depending on the type of electronic device. In various embodiments, the electronic apparatus may include at least one of the above-described elements. Some of the above-described elements may be omitted from the electronic device, or the electronic device may further include additional elements. Further, some of the elements of the electronic device according to various embodiments may be combined to constitute a single entity, and thus may equivalently execute functions of the corresponding elements before being combined.

The embodiments disclosed herein are provided merely to describe technical details of the disclosure and help the understanding of the disclosure, and do not limit the scope of the disclosure. Therefore, it should be construed that the scope of the disclosure includes all modifications and changes or various other embodiments based on the technical idea of the disclosure.

What is claimed is:

1. An electronic device comprising:
a sensor configured to acquire a biometric signal; and
a processor,
wherein the processor is configured to:
acquire a biometric signal by using the sensor;
measure first biometric information at least on the basis of a first portion of the biometric signal;
measure second biometric information at least on the basis of a second portion which is at least a part of the first portion; and
display at least one piece of information among the first biometric information and the second biometric information, on a display functionally connected to the electronic device.

2. The electronic device of claim 1, wherein the processor is configured to determine the second biometric information capable of being measured simultaneously with the first biometric information at least on the basis of at least one attribute related to measurement of the first biometric information.

3. The electronic device of claim 2, wherein the processor is configured to verify a measurement time for the measurement of the first biometric information, as at least one element among the at least one attribute.

4. The electronic device of claim 2, wherein the processor is configured to verify a type of light of the sensor for the measurement of the first biometric information, as at least one element among the at least one attribute.

5. The electronic device of claim 2, wherein the processor is configured to verify a frequency band of the biometric signal for the measurement of the first biometric information, as at least one element among the at least one attribute.

6. The electronic device of claim 1, wherein the processor is configured to display the second biometric information on the display when measurement of the second biometric information is completed.

7. The electronic device of claim 6, wherein the processor is configured to measure the first biometric information by using the measured second biometric information.

8. The electronic device of claim 1, wherein the processor is configured to:
make a determination of whether measurement of the first biometric information has been completed; and
continuously measure the second biometric information on the basis of at least a part of the biometric signal according to a result of the determination.

9. The electronic device of claim 1, wherein the processor is configured to:
when acquisition of the biometric signal from the sensor is stopped,
make a determination of whether measurement of the first biometric information and measurement of the second biometric information have been completed; and
display the first biometric information or the second biometric information measured until acquisition of the biometric signal is stopped, on the display at least on the basis of a result of the determination.

10. The electronic device of claim 1, wherein the processor is configured to:
when acquisition of the biometric signal from the sensor is stopped,
verify a time until acquisition of another biometric signal through the sensor;
when the verified time satisfies a designated condition,
measure the first biometric information or the second biometric information by using the biometric signal and another biometric signal; and
when the verified time does not satisfy the designated condition,
measure the first biometric information or the second biometric information by using another biometric signal.

11. An operating method of an electronic device, the operating method comprising:
acquiring a biometric signal by using a sensor of the electronic device;
measuring first biometric information at least on the basis of a first portion of the biometric signal, and measuring second biometric information at least on the basis of a second portion which is at least a part of the first portion; and
displaying at least one piece of information among the first biometric information and the second biometric information, on a display functionally connected to the electronic device.

12. The operating method of claim 11, wherein the measuring of the first biometric information at least on the basis of the first portion of the biometric signal, and the measuring of the second biometric information comprise determining the second biometric information capable of being measured simultaneously with the first biometric information at least on the basis of at least one attribute related to measurement of the first biometric information.

13. The operating method of claim 12, wherein the determining of the second biometric information comprises verifying a measurement time for the measurement of the first biometric information, as at least one element among the at least one attribute.

14. The operating method of claim 12, wherein the determining of the second biometric information comprises verifying a type of light of the sensor for the measurement of the first biometric information, as at least one element among the at least one attribute.

15. The operating method of claim 12, wherein the determining of the second biometric information comprises verifying a frequency band of the biometric signal for the measurement of the first biometric information, as at least one element among the at least one attribute.
